SEQUENCE LISTING (24 sequences)

SEQ ID NO: 1 - DNA, 20 bp, synthetic construct
  source 1..20: mol_type = other DNA
tgaggacccgtgtagagagg

SEQ ID NO: 2 - DNA, 21 bp, synthetic construct
  source 1..21: mol_type = other DNA
tgtgcgtgactacatacaagg

SEQ ID NO: 3 - DNA, 20 bp, synthetic construct
  source 1..20: mol_type = other DNA
gcttagtgaggaggctgtgg

SEQ ID NO: 4 - DNA, 23 bp, synthetic construct
  source 1..23: mol_type = other DNA
tgtgcgtgactacatacaaggta

SEQ ID NO: 5 - DNA, 20 bp, synthetic construct
  source 1..20: mol_type = other DNA
tttctgagcctcggtttccc

SEQ ID NO: 6 - DNA, 20 bp, synthetic construct
  source 1..20: mol_type = other DNA
aacggctgaggctactgaag

SEQ ID NO: 7 - DNA, 20 bp, synthetic construct
  source 1..20: mol_type = other DNA
tcggtttccctgtctgttgg

SEQ ID NO: 8 - DNA, 20 bp, synthetic construct
  source 1..20: mol_type = other DNA
gaacggctgaggctactgaa

SEQ ID NO: 9 - DNA, 22 bp, synthetic construct
  source 1..22: mol_type = other DNA
tgtcgtgtaagttgctagtgct

SEQ ID NO: 10 - DNA, 18 bp, synthetic construct
  source 1..18: mol_type = other DNA
tgaaccgggacaggggtg

SEQ ID NO: 11 - DNA, 21 bp, synthetic construct
  source 1..21: mol_type = other DNA
tgtcgtgtaagttgctagtgc

SEQ ID NO: 12 - DNA, 17 bp, synthetic construct
  source 1..17: mol_type = other DNA
tgaaccgggacaggggt

SEQ ID NO: 13 - DNA, 18 bp, synthetic construct
  source 1..18: mol_type = other DNA
gctgcgggtactgactcg

SEQ ID NO: 14 - DNA, 19 bp, synthetic construct
  source 1..19: mol_type = other DNA
gccgtgatgaggtatggag

SEQ ID NO: 15 - DNA, 20 bp, synthetic construct
  source 1..20: mol_type = other DNA
gaggctgcgggtactgactc

SEQ ID NO: 16 - DNA, 24 bp, synthetic construct
  source 1..24: mol_type = other DNA
ggcttctcaaagagcctcaatttc

SEQ ID NO: 17 - DNA, 20 bp, synthetic construct
  source 1..20: mol_type = other DNA
atccacaccgcggaagaagg

SEQ ID NO: 18 - DNA, 24 bp, synthetic construct
  source 1..24: mol_type = other DNA
ccgtgatgaggtatggagagacaa

SEQ ID NO: 19 - DNA, 25 bp, synthetic construct
  source 1..25: mol_type = other DNA
tttccctgtctgttgggaggtaact

SEQ ID NO: 20 - DNA, 23 bp, synthetic construct
  source 1..23: mol_type = other DNA
cctgcgttcacacaggacagaac

SEQ ID NO: 21 - DNA, 24 bp, synthetic construct
  source 1..24: mol_type = other DNA
atgtgaagaggtgccttgtgtggt

SEQ ID NO: 22 - DNA, 29 bp, synthetic construct
  source 1..29: mol_type = other DNA
ttaaaaacccgcccataatttctcactgc

SEQ ID NO: 23 - DNA, 28 bp, synthetic construct
  source 1..28: mol_type = other DNA
gcaggctcatggggctttgtaggagcag

SEQ ID NO: 24 - DNA, 25 bp, synthetic construct
  source 1..25: mol_type = other DNA
gcacagcccgctgtacctgaggact

(19) United States
(12) Patent Application Publication
(10) Pub. No.: US 2026/0022418 A1
(43) Pub. Date: Jan. 22, 2026

(54) METHODS, COMPOSITIONS AND SYSTEMS FOR SEQUENCING PKD1

(71) Applicant: Laboratory Corporation of America Holdings, Burlington, NC (US)

(72) Inventors: Shanshan Zhou, Burlington, NC (US); Brianne Vignero, Burlington, NC (US)

(73) Assignee: Laboratory Corporation of America Holdings, Burlington, NC (US)

(21) Appl. No.: 19/237,686

(22) Filed: Jun. 13, 2025

Related U.S. Application Data
(60) Provisional application No. 63/659,739, filed on Jun. 13, 2024.

(51) Int. Cl. C12Q 1/6869; C12Q 1/48; C12Q 1/6809; C12Q 1/6813; C12Q 1/686; C12Q 1/6876; C40B 40/06; C40B 50/06

(52) U.S. Cl. CPC C12Q 1/6869; C12Q 1/48; C12Q 1/6809; C12Q 1/6813; C12Q 1/686; C12Q 1/6876; C12Y 207/07; C40B 40/06; C40B 50/06

(57) ABSTRACT

Disclosed herein is a method for sequencing a PKD1 gene. The method may comprise amplifying exons 1-46 of the PKD1 gene using long-range PCR to form a plurality of long-range PCR products; and sequencing the plurality of long-range PCR products. Alternatively, the method may comprise amplifying exons 1-33 of the PKD1 gene using long-range PCR to form a plurality of long-range PCR products; amplifying exons 34-46 of the PKD1 gene by target-enrichment PCR to form a plurality of target-enrichment PCR products; and sequencing the plurality of long-range PCR products and the plurality of target-enrichment PCR products. Also disclosed herein are compositions, kits, and systems comprising primers and/or probes for performing the methods recited herein.

### CROSS-REFERENCE TO RELATED APPLICATIONS

This application claims benefit of priority to U.S. Provisional Application No. 63/659,739, filed Jun. 13, 2024, the entirety of which is incorporated herein by reference.

### REFERENCE TO A “SEQUENCE LISTING,” A TABLE, OR A COMPUTER PROGRAM LISTING APPENDIX SUBMITTED AS AN ASCII TEXT FILE

This application includes a Sequence Listing as a text file named “057618-1509625.xml” created Sep. 12, 2015 and containing 22,290 bytes, machine format IBM-PC, MS-Windows operating system. The material contained in this text file is incorporated by reference in its entirety for all purposes.

### FIELD OF THE INVENTION

Disclosed are methods, compositions, and systems for sequencing the Polycystin 1, Transient Receptor Potential Channel Interacting (PKD1) gene.

### BACKGROUND

PKD1 gene screening is challenging due to size (46 exons), allelic heterogeneity, high GC content, and the presence of six (6) PKD1 pseudogenes that are highly homologous to the sequence of the PKD1 gene. Proper amplification of the exons of PKD1 requires avoiding pseudogenes. Disclosed are methods, compositions, kits, and systems for improving the screening of PKD1 gene and avoiding pseudogenes.

### SUMMARY

Disclosed herein are methods for sequencing the PKD1 gene. The method may comprise amplifying exons 1-46 of a PKD1 gene using long-range PCR to form a plurality of long-range PCR products; and sequencing the plurality of long-range PCR products. Optionally, the method further comprises purifying, pooling, shearing, and amplifying the plurality of long-range PCR products prior to the sequencing step. The long-range PCR may comprise contacting the PKD1 gene with a plurality of primers comprising the nucleic acid sequences of SEQ ID NOs: 9-10, 15-16, and 19-24. Optionally, the long-range PCR comprises amplifying exons 1-46 of the PKD1 gene as five fragments. In certain embodiments, the first fragment corresponds to exon 1 of the PKD1 gene, the second fragment corresponds to exons 2-13 of the PKD1 gene, the third fragment corresponds to exons 14-21 of the PKD1 gene, the fourth fragment corresponds to exons 22-34 of the PKD1 gene, and the fifth fragment corresponds to exons 35-46 of the PKD1 gene. Optionally, the method primarily amplifies non-pseudogene forms of the PKD1 gene.

Alternatively, the method may comprise amplifying exons 1-33 of the PKD1 gene using long-range PCR to form a plurality of long-range PCR products; amplifying exons 34-46 of the PKD1 gene by target-enrichment PCR to form a plurality of target-enrichment PCR products; and sequencing the plurality of long-range PCR products and the plurality of target-enrichment PCR products. The long-range PCR of exons 1-33 may comprise amplifying exons 1-33 of the PKD1 gene as four fragments. Optionally, the method further comprises purifying, pooling, shearing, and amplifying the plurality of long-range PCR products prior to the sequencing step. The method may further comprise purifying, shearing, and amplifying the target-enrichment PCR products prior to the sequencing step. The target-enrichment PCR may comprise contacting the PKD1 gene with a plurality of probes that hybridize to exons 34-46; and purifying the hybridized exons 34-46. Optionally, the plurality of probes comprise nucleic acid sequences corresponding the chromosome coordinates of Table 11. Optionally, the plurality of primers and probes primarily amplify non-pseudogene forms of the PKD1 gene. Also disclosed herein are compositions, kits, and for performing the methods recited herein.

### BRIEF DESCRIPTION OF THE FIGURES

The present invention may be better understood by referring to the following non-limiting figures.

FIG. 1 shows a workflow of the long-range PCR amplification method for exons 1-46 of the PKD1 gene, in accordance with an embodiment of the disclosure.

FIG. 2 shows a workflow of the long-range PCR amplification method for individual fragments (1-5) corresponding to exons 1-46 of the PKD1 gene, in accordance with an embodiment of the disclosure.

FIG. 3 shows a workflow of the dual long-range and target-enrichment amplification and sequencing assay for sequencing the PKD1 gene, in accordance with an embodiment of the disclosure.

FIG. 4 shows a system for sequencing PKD1 in accordance with an embodiment of the disclosure.

FIG. 5 shows a block diagram of an analysis system used for PKD1 gene sequencing, in accordance with an embodiment of the disclosure.

FIG. 6 shows a workflow utilizing a system of the long-range PCR amplification, sequencing, and variant analyses for exons 1-46 of the PKD1 gene, in accordance with an embodiment of the disclosure.

FIG. 7 shows a Western Blot of the different exon fragments for the dual long-range and target-enrichment amplification method, in accordance with an embodiment of the disclosure.

### DETAILED DESCRIPTION

### Definitions

Unless otherwise defined herein, scientific and technical terms used in connection with the present invention shall have the meanings that are commonly understood by those of ordinary skill in the art. Further, unless otherwise required by context, singular terms shall include pluralities, and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. Known methods and techniques are generally performed according to conventional methods well known in the art and as described in various general and more specific references that are discussed throughout the present specification unless otherwise indicated. Enzymatic reactions and purification techniques are performed according to manufacturer's specifications, as commonly accomplished in the art or as described herein. The nomenclatures used in connection with the laboratory procedures and techniques described herein are those well-known and commonly used in the art.

The following terms, unless otherwise indicated, shall be understood to have the following meanings:

As used herein, the terms “a”, “an”, and “the” can refer to one or more unless specifically noted otherwise.

The use of the term “or” is used to mean “and/or” unless explicitly indicated to refer to alternatives only or the alternatives are mutually exclusive, although the disclosure supports a definition that refers to only alternatives and “and/or.” As used herein “another” can mean at least a second or more.

Throughout this application, the term “about” is used to indicate that a value includes the inherent variation of error for the device, the method being employed to determine the value, or the variation that exists among samples. In certain embodiments, the term “about” includes values that are ±10% of the indicated value.

As used herein, a “biological sample” refers to a sample of tissue or fluid isolated from a subject including, but not limited to, for example, blood, plasma, serum, fecal matter, urine, bone marrow, bile, spinal fluid, lymph fluid, samples of the skin, external secretions from the body, such as from skin, respiratory, intestinal and genitourinary tracts, tears, saliva, milk, blood cells, organs, biopsies and also in vitro cell culture constituents, for example conditioned media resulting from the growth of cells and tissues in culture medium, for example recombinant cells and cell components.

As used herein, the term “comprising” is intended herein to be open-ended, including not only the recited elements, but further encompassing any additional elements. The term “comprising” is intended to encompass the term “consisting of”.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “label” refers to a molecule, or moiety thereof, that provides a detectable characteristic. The detectable characteristic can be, for example, an optical signal such as absorbance of radiation, fluorescence emission, luminescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like.

As used herein, the term “nucleotide” can be used to refer to a native nucleotide or analog thereof. Examples include, but are not limited to, nucleotide triphosphates (NTPs) such as ribonucleotide triphosphates (rNTPs), deoxyribonucleotide triphosphates (dNTPs), or non-natural analogs thereof such as dideoxyribonucleotide triphosphates (ddNTPs) or reversibly terminated nucleotide triphosphates (rtNTPs).

A “modified nucleotide” or “edited nucleotide” refers to a nucleotide sequence of interest that comprises at least one alteration when compared to its non-modified nucleotide sequence. Such “alterations” include, for example: substitution of at least one nucleotide, a deletion of at least one nucleotide, an insertion of at least one nucleotide, or any combination thereof.

As used herein, the term “polymerase” can be used to refer to a nucleic acid synthesizing enzyme, including but not limited to, DNA polymerase, RNA polymerase, reverse transcriptase, primase and transferase. Typically, the polymerase has one or more active sites at which nucleotide binding and/or catalysis of nucleotide polymerization may occur. The polymerase may catalyze the polymerization of nucleotides to the 3′ end of the first strand of the double stranded nucleic acid molecule. For example, a polymerase catalyzes the addition of a next correct nucleotide to the 3′ oxygen group of the first strand of the double stranded nucleic acid molecule via a phosphodiester bond, thereby covalently incorporating the nucleotide to the first strand of the double stranded nucleic acid molecule. Optionally, a polymerase need not be capable of nucleotide incorporation under one or more conditions used in a method set forth herein. For example, a mutant polymerase may be capable of forming a ternary complex but incapable of catalyzing nucleotide incorporation.

As used herein, the term “primer” refers to a nucleic acid having a sequence that binds to a nucleic acid at or near a template sequence. Generally, the primer binds in a configuration that allows replication of the template, for example, via polymerase extension of the primer. The primer can be a first portion of a nucleic acid molecule that binds to a second portion of the nucleic acid molecule, the first portion being a primer sequence and the second portion being a primer binding sequence (e.g. a hairpin primer). Alternatively, the primer can be a first nucleic acid molecule that binds to a second nucleic acid molecule having the template sequence. A primer can consist of DNA, RNA or analogs thereof. A primer can have an extendible 3′ end, a 3′ end that is blocked from primer extension or a 3′ end that is capped to hinder or preclude ternary complex formation. Genomic DNA (gDNA) can be used as the template DNA sequence.

As used herein, “polymerase chain reaction (PCR)” is a method used for amplification of nucleotide sequences. During a single cycle of PCR amplification, a double-stranded target DNA sequence is denatured; primers are annealed to each strand of the denatured target; and the primers are extended by a DNA polymerase. This cycle is repeated, generally between 25 and 40 times, in order to concentrate the number of copies of a target DNA sequence in a sample. The primers used in PCR are designed to anneal to the denatured target DNA sequence strands in a position and orientation such that the extended primers are complementary copies of the target DNA sequences. On subsequent amplification cycles, the extended primers can also serve as targets for amplification.

As used herein, the terms “subject”, “patient” and “individual” are used interchangeably. A subject may be any mammal, including a human.

As used herein, “touchdown PCR” refers to a technique in which the first annealing temperature is set to a relatively high temperature and the annealing temperature is gradually reduced for each cycle, and, midway and thereafter, PCR is performed in the same manner as general PCR.

As used herein, “long-range PCR” is a technique used to amplify significantly longer DNA fragments than those amplified in traditional PCR techniques. For example, long-range PCR may use polymerases capable of amplifying DNA fragments up to 60 kb long. Generally, long-range PCR utilizes amplification conditions which improve target strand denaturation (e.g., higher denaturation temperatures, addition of cosolvents), and which protect DNA from degradation; utilizes longer elongation times; and minimizes incorporation of erroneous nucleotides by utilizing polymerases having high processivity to reduce mismatches, thereby enabling amplification of extended strands of DNA.

As used herein, “target-enrichment PCR” or “target-enriched PCR” is a technique that employs hybridization of an isolated DNA probe complimentary to a DNA target sequence(s) to enrich the target sequence(s) prior to amplification by traditional (i.e., not long-range) PCR.

Various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The embodiments set forth below and recited in the claims can be understood in view of the above definitions.

### Methods

Disclosed herein are methods for sequencing a PKD1 gene. Optionally, the PKD1 gene sequenced in the methods herein is in a genomic DNA (gDNA) sample. For any of the methods, the DNA may be isolated from a biological sample from a subject. In certain embodiments, the biological sample may be tissue or blood, or a blood product such as plasma or serum. Optionally, the PKD1 gene sequenced in the methods herein is in a genomic DNA (gDNA) sample extracted from blood. Or other biological samples may be used. The DNA can be readily obtained from the biological sample using a commercially available DNA extraction kit or the like.

The method may comprise amplifying exons 1-46 of the PKD1 gene using long-range PCR to form a plurality of long-range PCR products; and sequencing the plurality of long-range PCR products. Optionally, the method comprises purifying the plurality of long-range PCR products prior to the sequencing step. The method may optionally comprise pooling the plurality of long-range PCR products prior to the sequencing step. In some embodiments, the method may comprise shearing the plurality of long-range PCR products prior to the sequencing step. Optionally, the method comprises amplifying the plurality of long-range PCR products prior to the sequencing step. The method may in some cases, further comprise creating a library of the amplified long-range PCR products. The library may then be purified and sequencing of the long-range PCR products performed. For example, as described in more detail herein, pooled and purified long-range PCR amplicons may be sheared, and the sheared fragments may optionally be subjected to end-repair, and/or addition of a poly(dA) tail and/or other adapters, and after an optional additional purification step, amplified by standard (i.e., not long-range) PCR to generate a library for sequencing.

Optionally, the long-range PCR comprises contacting the PKD1 gene with a plurality of primers comprising the nucleic acid sequences of SEQ ID NOs: 9-10, 15-16, and 19-24. Optionally, the long-range PCR comprises amplifying exons 1-46 of the PKD1 gene as five fragments. Optionally, the first fragment corresponds to exon 1 of the PKD1 gene, the second fragment corresponds to exons 2-13 of the PKD1 gene, the third fragment corresponds to exons 14-21 of the PKD1 gene, the fourth fragment corresponds to exons 22-34 of the PKD1 gene, and the fifth fragment corresponds to exons 35-46 of the PKD1 gene.

Alternatively, the method may comprise amplifying exons 1-33 of the PKD1 gene using long-range PCR to form a plurality of long-range PCR products; amplifying exons 34-46 of the PKD1 gene by target-enrichment PCR to form a plurality of target-enrichment PCR products; and sequencing the plurality of long-range PCR products and the plurality of target-enrichment PCR products. Optionally, the long-range PCR and target-enrichment PCR is performed concurrently.

Optionally, the method comprises purifying the plurality of long-range PCR products prior to the sequencing step. Optionally, the method comprises pooling the plurality of long-range PCR products prior to the sequencing step. Optionally, the method comprises shearing the plurality of long-range PCR products and/or the plurality of target-enrichment PCR products prior to the sequencing step. Optionally, the method comprises amplifying the plurality of sheared long-range PCR products and/or the plurality of target-enrichment PCR products prior to the sequencing step. Optionally, the method comprises creating a library of the amplified long-range PCR products and/or the plurality of target-enrichment PCR products. The library may then be purified and sequencing of the long-range PCR products performed.

Where long-range PCR and target-enrichment PCR are performed, the long-range PCR of exons 1-33 may be performed in a manner similar to that described above for exons 1-46. Thus, exons 1-33 may be amplified by long-range PCR, using primers that generate amplicons for four fragments. Optionally, the long-range PCR comprises contacting the PKD1 gene with a plurality of primers comprising the nucleic acid sequences of SEQ ID NOs: 1-18. Optionally, the first fragment corresponds to exon 1 of the PKD1 gene, the second fragment corresponds to exons 2-13 of the PKD1 gene, the third fragment corresponds to exons 14-21 of the PKD1 gene, the fourth fragment corresponds to exons 22-33 of the PKD1 gene. The long-range PCR fragments may then be purified and/or pooled together. At this point the purified pooled long-range PCR amplicons may be sheared, the sheared fragments may optionally be subjected to end-repair, addition of a poly(dA) tail and/or other adapters, and after an optional additional purification step, amplified by standard (i.e., not long-range) PCR to generate a library for sequencing.

Optionally, the target-enrichment PCR may then comprise contacting the library with a plurality of probes, wherein exons 34-46 hybridize to the plurality of probes; and purifying the hybridized exons 34-46. Thus, in certain embodiments, genomic DNA may be isolated. The isolated DNA may then be sheared and the sheared DNA subjected to subjected to end-repair, addition of a poly(dA) tail and/or other adapters, and after an optional additional purification step, amplified by standard (i.e., not long-range) PCR to generate a library. Optionally, the target-enrichment PCR may then comprise contacting the library with a plurality of probes, wherein exons 34-46 hybridize to the plurality of probes; and purifying the hybridized exons 34-46. Optionally, the plurality of probes comprise the nucleic acid sequences corresponding the chromosome coordinates of Table 11.

For both long-range and target-enrichment PCR, the PCR cycling conditions are not particularly limited as long as the desired exons of PKD1 can be amplified under conditions to minimize amplification of any PKD1 pseudogene sequences. In other words, the methods provided herein primarily amplify non-pseudogene forms of the PKD1 gene such that pseudogene forms of the PKD1 gene are not detectable. The primers used in the methods provided herein were designed in genomic regions containing mismatches to PKD1 pseudogenes, and the bioinformatics processes used herein filter the read pairs mapping to both the pseudogene and PKD1 gene target.

### Long-Range PCR

For both long-range amplification of exons 1-33 and/or 1-46, long-range PCR amplification may be used. The test sample (e.g., gDNA) and the desired primers can initially be mixed in an amplification reaction mixture containing reagents necessary for amplification of the target DNA sequence (e.g., nucleotides, enzyme buffers, etc.). The amplification reaction mixture, including the test sample and primers, is then subjected to PCR. Where a commercial kit and/or enzyme is used, the manufacturer's suggested protocol can be used for amplifying the target (i.e., the PKD1 exons of interest) DNA.

In certain embodiments, long-range PCR is performed using a touchdown PCR as touchdown PCR can inhibit non-specific amplification (see the Examples below). In an embodiment, touchdown PCR protocol utilizes multiple iterations of a three-step cycle, each cycle comprising: first, melting the template DNA at a melting temperature (TM)—for example 98° C.; second, allowing primers to anneal at an annealing temperature, for example 70° C.; and third, allowing polymerase to extend DNA at an elongation temperature for example, 690° C. The annealing temperature may be the same as the elongation temperature, or annealing temperature may be the different from the elongation temperature. In the first cycle, a high annealing temperature is used—for example 70° C. In each subsequent cycle of touchdown PCR, the annealing temperature is incrementally decreased until the lowest annealing temperature at or slightly below the optimal annealing temperature is reached. In an embodiment, the annealing temperature is decreased by an increment of less than or equal to 0.1, 0.2, 0.3, 0.4, 0.5, 0.6, 0.7, 0.8, 0.9, 1.0, 1.1, 1.2, 1.3, 1.4, 1.5, 1.61.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0° C. in each cycle until the bottom annealing temperature is reached—for example 50° C. In an embodiment, the annealing temperature is decreased by 1° C. Once the bottom annealing temperature is reached, one or more cycles are performed using the bottom annealing temperature.

For long-range PCR of exons 1-46 or exons 1-33, the initial thermal denaturation temperature can be set to 95 to 100° C., and preferably 97 to 99° C. In an embodiment, the initial thermal denaturation temperature is 98° C. The initial thermal denaturation time can be set to, for example, 60 to 300 seconds, and preferably 120 to 240 seconds. In an embodiment, the initial thermal denaturation time is 180 seconds. The second thermal denaturation can be set to 95 to 100° C., and preferably 97 to 99° C. In an embodiment, the second thermal denaturation temperature is 98° C. The second thermal denaturation time can be set to, for example, 5 to 20 seconds, and preferably 10 seconds. In an embodiment, the second thermal denaturation time is 10 seconds. The annealing step can be performed using touchdown PCR. The starting annealing temperature for hybridizing primers can be set to, for example, 65 to 75° C., and preferably 68 to 70° C. In an embodiment, the starting annealing temperature is 70° C. The annealing time can be set to, for example, 5 to 30 seconds, and preferably 10 to 20 seconds. In an embodiment, the annealing time is 15 seconds. The touchdown temperature can be set to decrease by 1 to 3° C. for each PCR cycle, and preferably is set to decrease by 1° C. per cycle. The final annealing temperature can be 45 to 60° C., and preferably 50 to 55° C. In an embodiment, the final annealing temperature is 50° C. The initial elongation reaction temperature can be set to, for example, 65 to 75° C., and preferably 67 to 72° C. In an embodiment, the initial elongation reaction temperature is 69° C. The initial elongation reaction time can be set to, for example, 5 to 15 minutes, and preferably 8 to 12 minutes. In an embodiment, the initial elongation reaction time is 10 minutes.

The number of amplification cycles can be repeated until the required amounts of amplification products are obtained. For example, the number of cycles can be 10 to 40, and preferably about 20 to 35. Following amplification, the final elongation reaction temperature can be set to, for example, 65 to 75° C., and preferably 67 to 72° C. In an embodiment, the final elongation reaction temperature is 69° C. The final elongation reaction time can be set to, for example, 5 to 15 minutes, and preferably 8 to 12 minutes. In an embodiment, the initial elongation reaction time is 10 minutes. In an embodiment, the PCR cycling conditions include one of, any combination of, or all of the conditions with respect to the temperature and time of each thermal denaturation, annealing, and elongation reaction of PCR and the number of cycles.

The reaction volume may be varied as necessitated by the sample quality or other reaction conditions. Thus, reaction volumes ranging from 10 to 1,000 μL, or 20 to 200 μL, or 30 to 100 μL may be used. In an embodiment, a 50 μL reaction mixture can be used. For example, the reaction mixture can be from 25 to 50 μL, from 30 to 55 μL, or from 35 to 60 μL, from 30 to 35 μL, from 35 to 40 μL, from 40 to 45 μL, from 45 to 50 μL, from 50 to 55 μL, or from 55 to 60 μL.

In certain embodiments, the primers for long-range PCR may amplify exons 1-46, exons 1-33 or another subset of exons 1-46. The primers may be specifically designed to amplify non-pseudogene forms of the PKD1 gene exons. In certain embodiments, the primers may comprise SEQ ID NOs: 1-24 as disclosed herein. In certain embodiments, the primers comprise SEQ ID NOS: 15 and 16 for amplification of PKD1 exon 1 by long-range PCR, and/or SEQ ID NOs: 9 and 10 for amplification of PKD1 exons 2-13 by long-range PCR, and/or SEQ ID NOs: 19 and 20 for amplification of PKD1 exons 14-21 by long-range PCR, and/or SEQ ID NOs: 21 and 22 for amplification of PKD1 exons 22-34 by long-range PCR, and/or SEQ ID NOs: 23 and 24 for amplification of PKD1 exons 35-46 by long-range PCR. Or the primers may comprise SEQ ID NOs: 1 and 2 and/or SEQ ID NOs: 3 and 4 for long-range PCR of PKD1 exons 22-33, and/or SEQ ID NOs: 5 and 6 and/or SEQ ID NOs: 7 and 8 for long-range PCR of PKD1 exons 14-21, and/or SEQ ID NOs: 9 and 10 and/or SEQ ID NOs: 11 and 12 for long-range PCR of PKD1 exons 2-13, and/or SEQ ID NOs: 12 and 13 and/or SEQ ID NOs: 15 and 16 and/or SEQ ID NOs: 17 and 18 for long-range PCR of PKD1 exons 22-33.

Following the long-range PCR amplification reaction, the presence or absence of detectable PCR (i.e., amplification) products may be determined. In an embodiment, analysis of the PCR products includes determining the size of any detectable PCR products. The PCR products can be detected by a variety of methods including, but not limited to, gel electrophoresis (e.g., agarose or acrylamide gel electrophoresis) or HPLC, followed by detection of the size fractionated amplification products by methods such as staining, or hybridization of labeled probes. In an embodiment, ethidium bromide agarose gel electrophoresis is used.

Once the PCR products are amplified, the amplicons can be pooled together with about or at equal molarity. At this point impurities may be removed by a first purification step using e.g., magnetic beads, gel extraction, or other known purification methods.

After the optional purification step, the pooled amplicons may be sheared with an enzyme mix and any nicks repaired. Next, the pooled fragments may be tailed, e.g., by addition of poly(dA) tails. As shown in the Examples, poly(dA) tailing may be performed at the same time as enzyme fragmentation and end repair using a master mix and specific cycling. At this point, adapter(s) may be added and the product amplified (e.g., using a universal primer(s)). In an embodiment, a second purification step is performed after tailing using e.g., magnetic beads, gel extraction methods, or other known purification methods. For example, for magnetic bead purification, at least one of the adapters ligated to the target nucleic acid fragments may contain a first binding member that can bind to a second binding member conjugated to the magnetic beads such that the magnetic beads can capture the adapter-ligated nucleic acid fragments. The first binding member and the second binding member are referred to as a binding pair. The nucleic acid fragments can then be washed and purified. At this point, the purified products (i.e., the library) are prepared for quality control, and sequenced.

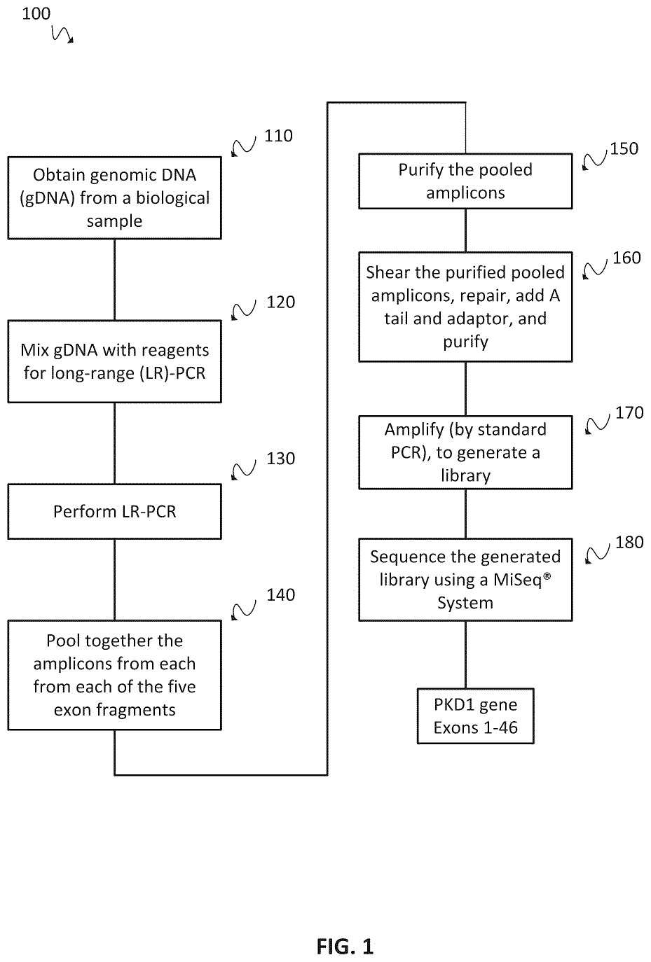

An embodiment of a long-range PCR method (100) for sequencing PKD1 exons 1-46 is shown in FIG. 1. Thus, in an embodiment genomic DNA is obtained (110). The genomic DNA may be obtained from a variety of biological samples. In certain embodiments, the sample is blood, a blood product e.g., plasma or serum, or tissue. Next, the DNA is mixed with reagents for PCR amplification (120) and PCR is performed (130). In certain embodiments, the primer pairs are designed for amplification by long-range PCR. In an embodiment, long-range PCR is performed such that a first amplicon corresponds to exon 1, a second amplicon corresponds to exons 2-13, a third amplicon corresponds to exons 14-21, a fourth amplicon corresponds to exons 22-34, and a fifth amplicon corresponds to exons 35-46. In an embodiment, the PCR is touchdown PCR. Next, the amplicons from each fragment for the different primer pairs may be pooled together (140). The pooled amplicons may then be purified, using a purification method such as magnetic beads (150). The purified pooled amplicons may then be sheared mechanically or digested into smaller fragments with an enzyme (e.g., a restriction enzyme) and repaired (160). At this point, a poly(dA) tail and adapters are added to the sheared pooled amplicons (160). Next, the sheared pooled amplicons are further amplified by standard PCR to generate a library (170). In an embodiment, the library is sequenced using, e.g., a MiSeq® System (180).

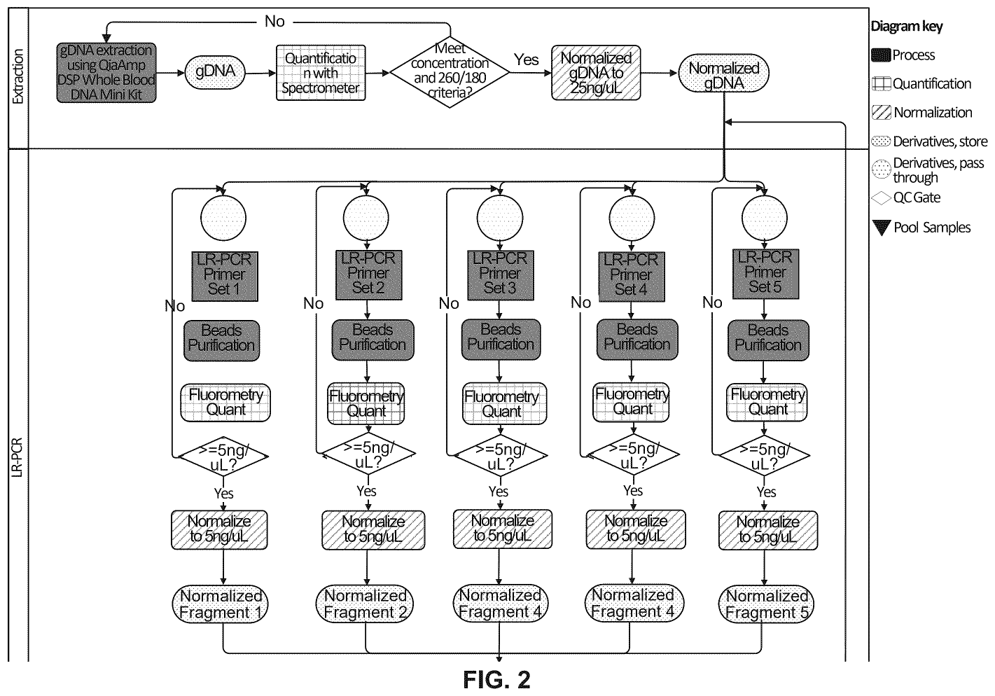

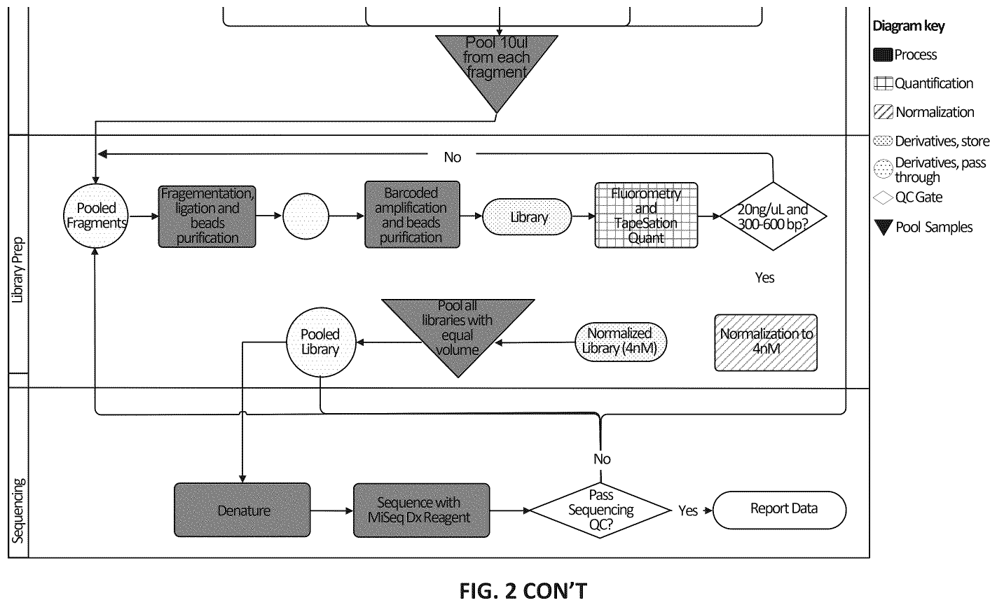

FIG. 2 shows an example workflow for long-range PCR amplification and analysis of the PKD1 gene including quality control steps that may be performed. As shown in FIG. 2, each of the steps of the method may be subjected to a quality control evaluation. Steps that do not pass quality control may be repeated until product of sufficient quality and quantity is obtained. Thus, steps subjected to quality control include isolation of gDNA template, long-range PCR to generate 5 fragments corresponding to exons 1-46 of the PKD1 gene, generation of libraries corresponding to the pooled fragments, and a determination that the pooled libraries are suitable for sequencing.

Thus, as shown in FIG. 2, long-range PCR of gDNA is performed for 5 fragments corresponding to exons 1-46 of the PKD1 gene. As noted herein, the first fragment may correspond to exon 1 of the PKD1 gene, the second fragment may correspond to exons 2-13 of the PKD1 gene, the third fragment may correspond to exons 14-21 of the PKD1 gene, the fourth fragment may correspond to exons 22-34 of the PKD1 gene, and the fifth fragment may correspond to exons 35-46 of the PKD1 gene. In certain embodiments, the primers used are those as described herein.

After long-range PCR, the amplicons are purified (e.g., using beads) and quantified and then, if sufficient amounts of the amplicons obtained, the amplicons corresponding to the 5 fragments are normalized and pooled at approximately equal molarity. The pooled amplicons are sheared with an enzyme mix and any nicks repaired. Next, the pooled fragments may be tailed, e.g., by addition of poly(dA) tails, adapter(s) are added and the product amplified (e.g., using a universal primer(s)) and a library of amplicons prepared. As illustrated in FIG. 2, the library is then evaluated for amplicons having the correct size and sufficient amounts. At this point libraries may be normalized and further pooled for sequencing. If sequencing does not pass quality control, the process, or certain steps of the process may be repeated.

### Target-Enrichment PCR

In certain embodiments, target-enrichment PCR is used for analysis of a subset of exons 1-46. In certain embodiments, target-enrichment PCR may be used for analysis of exons 34-46.

As with long-range PCR, the template for target-enrichment PCR may be genomic DNA. The genomic DNA may be sheared or digested using, e.g., a restriction enzyme, and any nicks in double stranded DNA (dsDNA) may be repaired. The digested/sheared DNA may then be amplified by PCR as described herein. At this point, the resultant PCR products may be tailed (e.g., poly(dA)) and, following tailing, an adapter may be attached. The tailed and adapter-ligated fragments may then be amplified using a universal primer, followed by an optional purification step. The purification step may comprise using magnetic beads, gel extraction methods, or other purification methods known in the art.

In an embodiment, the purified amplification products are then enriched by hybridization to sequence-specific probes. Examples of sequences targeted by PKD1 sequence-specific probes are disclosed herein (Table 11). In an embodiment, the PKD1 gene is contacted with a plurality of probes comprising nucleic acid sequences corresponding the chromosome coordinates of Table 11. In an embodiment, the purified products are allowed to hybridize with a probe panel. The probe panel may be purchased from a commercial source. The hybridization of the purified products with the probe panel can take place over at least 6 hours, at least 12 hours, at least 18 hours, at least 24 hours, at least 32 hours, or at least 48 hours. Preferably, the hybridization step takes place overnight. After hybridization, the hybridized products may be washed to remove unbound nucleic acid and, optionally, subjected to an additional purification step. The additional purification step may comprise using magnetic beads, gel extraction methods, or other purification methods known in the art. Optionally, in some embodiments, the purified, hybridized products may undergo quality control and sequencing.

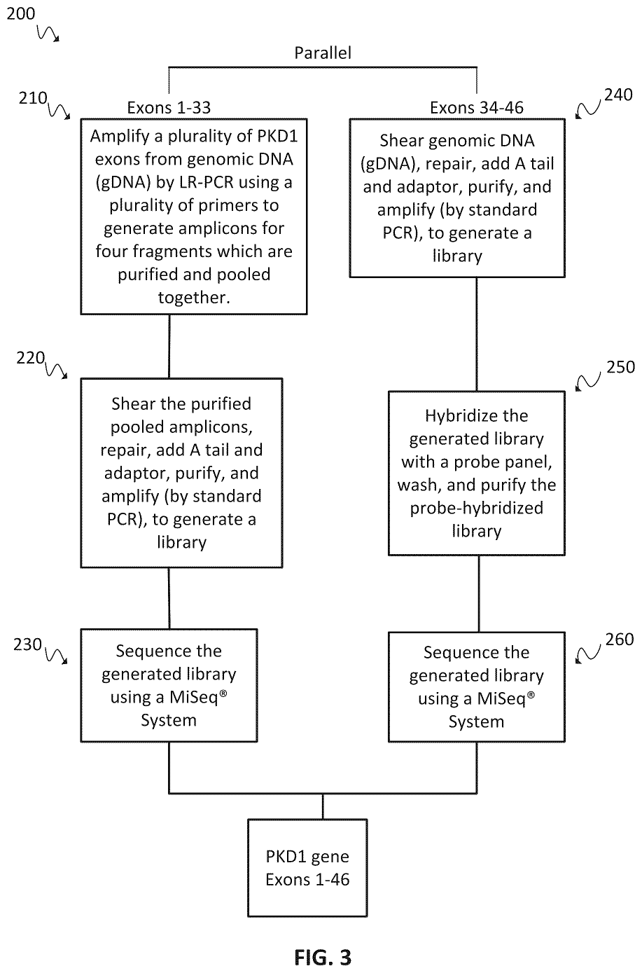

An embodiment of a dual method (300) for sequencing PKD1 exons 1-46 is shown in FIG. 3. Thus, in an embodiment, the method comprises the use of long-range PCR of PKD1 exons 1-33 and target-enrichment PCR of exons 34-46. The long-range PCR and target-enrichment PCR methods may be run concurrently. In an embodiment, four PKD1 exon fragments for exons 1-33 are amplified from genomic DNA by long-range PCR using a plurality of primers to generate amplicons for four fragments which are purified and pooled together (310). For example, exons 1-33 may be amplified as 4 fragments (amplicons), where one fragment includes exons 22-33, a second fragment includes exons 14-21, a third fragment includes exons 2-13 and a fourth fragment includes exon 1. The purified pooled amplicons may then be sheared mechanically or digested into smaller fragments with an enzyme (e.g., a restriction enzyme) and the fragment ends repaired (320). In an embodiment, a poly(dA) tail and adapters are added to the sheared, pooled amplicons after repair (320). The amplicons may then be amplified by standard PCR to generate a library (320). Optionally, the library may then be sequenced, e.g., using a MiSeq® System or other sequencing protocols (330).

In parallel to long-range PCR for exons 1-33, hybrid capture and amplification may be used to analyze exons 35-36. For example, genomic DNA may be sheared or digested with an enzyme mix, followed by repair of the ends of the fragmented DNA molecules (340). A poly(dA) tail and adapters may then be added to the sheared genomic DNA (340). The mixture may then be purified before further amplification by standard PCR to generate a library (340). The generated library may then be hybridized with a probe panel (350) corresponding to known targets in exons 34-46. The hybridized library may then be washed and purified (350) and sequenced, e.g., using a MiSeq® System or another sequencing method (360).

### Compositions, Kits, and Systems

Also disclosed herein are compositions comprising primers and/or probes for performing the methods recited herein. In certain embodiments, the compositions may be included in a kit. The kit may comprise the primers and/or probes in packaged form (e.g., either liquid or solid) and/or instructions for use. Optionally, also included as compositions and/or for inclusion in a kit are positive and negative control samples. Kits may further comprise computer-programmable products (e.g., software) as disclosed herein.

Thus, disclosed is a composition and/or kit comprising a plurality of primers long-range PCR of exons of the PKD1 gene. In certain embodiments, the primers for long-range PCR may amplify exons 1-46, exons 1-33 or another subset of exons 1-46. The primers may be specifically designed to amplify non-pseudogene forms of the PKD1 gene exons. In certain embodiments, the primers may comprise SEQ ID NOs: 1-24 as disclosed herein. In certain embodiments, the primers comprise SEQ ID NOS: 15 and 16 for amplification of PKD1 exon 1 by long-range PCR, and/or SEQ ID NOs: 9 and 10 for amplification of PKD1 exons 2-13 by long-range PCR, and/or SEQ ID NOs: 19 and 20 for amplification of PKD1 exons 14-21 by long-range PCR, and/or SEQ ID NOs: 21 and 22 for amplification of PKD1 exons 22-34 by long-range PCR, and/or SEQ ID NOs: 23 and 24 for amplification of PKD1 exons 35-46 by long-range PCR. Or the primers may comprise SEQ ID NOs: 1 and 2 and/or SEQ ID NOs: 3 and 4 for long-range PCR of PKD1 exons 22-33, and/or SEQ ID NOs: 5 and 6 and/or SEQ ID NOs: 7 and 8 for long-range PCR of PKD1 exons 14-21, and/or SEQ ID NOs: 9 and 10 and/or SEQ ID NOs: 11 and 12 for long-range PCR of PKD1 exons 2-13, and/or SEQ ID NOs: 12 and 13 and/or SEQ ID NOs: 15 and 16 and/or SEQ ID NOs: 17 and 18 for long-range PCR of PKD1 exons 22-33.

Also provided herein is a plurality of probes comprising the probes listed in Table 11. Optionally, the plurality of probes comprise the nucleic acid sequences corresponding the chromosome coordinates of Table 11.

Thus, disclosed is a composition and/or kit comprising at least one primer pair for conducting the method of sequencing a PKD1 gene from genomic DNA (gDNA), wherein the plurality of fragments are designed to amplify non-pseudogene forms of the PKD1 gene exons 1-46 for selective sequencing, wherein the plurality of primers target exons 1-46 for long-range PCR for enrichment. Optionally, the plurality of primers used for long-range PCR comprise the nucleic acid sequences of SEQ ID NOs: 9-10, 15-16, and 19-24. Where long-range PCR is used for exons 1-33 and target-enrichment PCR is used for exons 34-46, disclosed herein is a composition and/or kit comprising a plurality of primers and/or probes for conducting the method of sequencing a PKD1 gene from genomic DNA (gDNA), wherein a subset of the plurality of primers target exons 1-33 for long-range PCR and wherein a subset of the plurality of primers and the plurality of probes target exons 34-64 for target-enrichment PCR. Optionally, the plurality of primers used when long-range PCR is used for exons 1-33 comprise SEQ ID NOs: 1-18. Optionally, the plurality of probes comprise the nucleic acid sequence corresponding the chromosome coordinates of Table 11.

Optionally, the PKD1 gene sequenced in using the compositions and/or kits herein is in a genomic DNA (gDNA) sample. For any of the disclosed compositions and/or kits, the DNA may be isolated from a biological sample from a subject. The DNA can be readily obtained from a biological sample using a commercially available DNA extraction kit or the like. In certain embodiments, the biological sample may be a tissue sample or a blood sample (e.g., blood, serum or plasma). Or other biological samples may be used.

Additionally disclosed are systems for using the disclosed compositions and/or kits or performing the disclosed methods. Where long-range PCR is used for exons 1-46, disclosed herein is a system for sequencing a PKD1 gene, the system comprising: a component for amplifying exons 1-46 of the PKD1 gene by long-range PCR to form a plurality of long-range PCR products; and a component for sequencing the plurality of long-range PCR products.

Where long-range PCR is used for exons 1-33 and target-enrichment PCR is used for exons 34-46, disclosed herein is a system for sequencing a PKD1 gene, the system comprising: a component for amplifying exons 1-33 of the PKD1 gene by long-range PCR to form a plurality of long-range PCR products; a component for amplifying exons 34-46 of the PKD1 gene using target-enrichment PCR to form a plurality of target-enrichment PCR products; and a component for sequencing the plurality of long-range PCR products and the plurality of target-enrichment PCR products.

The systems may include additional components. For example, the system may further include a component for purifying the plurality of PCR products generated by long-range PCR amplification of exons 1-46 and/or exons 1-33. The system may also comprise a component for pooling the plurality of purified long-range PCR products to create a long-range PCR product mixture and/or shearing the pooled long-range PCR products to create a sheared PCR product mixture and/or a component or components for end-repair, and/or poly(dA)-tailing and/or addition of adapters to the sheared PCR product. The system may also comprise a component for PCR amplifying the pooled and/or sheared PCR product to create a library. The system may also comprise a component for purifying the library for sequencing. Methods of purifying, pooling, and shearing applicable to the present systems are described above and elsewhere herein.

Where target-enrichment PCR is used for analysis of exons 34-46, the system may include a component for shearing genomic DNA and/or a component subsequent repair of the sheared DNA and/or a component for PCR amplification of the sheared DNA. The system may also include a component for adding a poly(dA) tail and/or an adapter to the amplified PCR products. Also included may be a component for amplifying the tailed and adapter-ligated fragments using a universal primer, as well as a component for additional purification (e.g., using magnetic beads, gel extraction methods, or other purification methods known in the art). Methods of purifying and shearing applicable to the present systems are described above and elsewhere herein. The system may also include a component for hybridization of the PCR products to a plurality of probes. Examples of sequences targeted by PKD1 sequence-specific probes are disclosed herein (Table 11). Optionally, the plurality of probes comprise the nucleic acid sequences corresponding the chromosome coordinates of Table 11. The hybridization of the purified products with the probe panel can take place over at least 6 hours, at least 12 hours, at least 18 hours, at least 24 hours, at least 32 hours, or at least 48 hours. Preferably, the hybridization step takes place overnight. The system may also comprise a component for washing the hybridized products free of unbound nucleic acid as well as additional components for further purification (e.g., using magnetic beads, gel extraction methods, or other known purification methods as described herein.

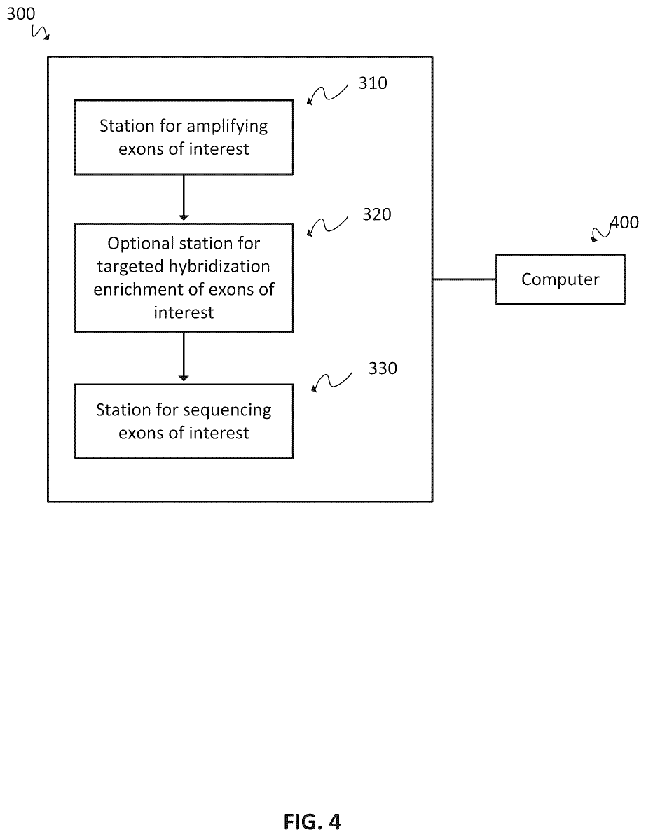

An embodiment of a system and/or kit (400) for sequencing PKD1 exons 1-46 is shown in FIG. 4. Thus, in an embodiment, the system may comprise a component for amplifying exons of interest (410), optionally using long-range PCR. Optionally, in an embodiment, the system may comprise a component for targeted hybridization enrichment of exons of interest (420). In an embodiment, the system may comprise a component for sequencing exons of interest (430).

In some embodiments, the system and/or kit further comprises a computer and/or a data processor (440). As disclosed herein, in certain embodiments, the system may comprise one or more computers, and/or a computer product tangibly embodied in a non-transitory computer readable storage medium containing instructions which, when executed on the one or more data processors, cause the one or more data processors to perform actions for performing the methods or implementing the systems and/or kits of any of the embodiments disclosed herein.

Thus, also disclosed is a computer program product and/or a computer system comprising instructions to perform any of the methods, or to use any of the compositions and/or kits, or to run any of the stations or components of the systems disclosed herein. Thus further disclosed is a system and/or computer-program product tangibly embodied in a non-transitory machine-readable storage medium for assessing amplification of at least one of the PKD1 exons, assessing the presence or absence of insertions, deletions or other changes, determining the sequence of a PKD1 gene from a biological sample. In certain embodiments, the system and/or computer program product may comprise components for detecting a PCR product. Also, the system and/or computer programmable product may comprise components to perform statistical analysis of the data. In some embodiments, this invention provides a system for sequencing a PKD1 gene from genomic DNA (gDNA) comprising one or more processors and non-transitory machine-readable storage medium and/or memory coupled to one or more processors, and the memory or the non-transitory machine readable storage medium encoded with a set of instructions configured to perform a process.

The systems and computer products may perform any of the methods disclosed herein. One or more embodiments described herein can be implemented using programmatic modules, engines, or components. A programmatic module, engine, or component can include a program, a sub-routine, a portion of a program, a software component, or a hardware component capable of performing one or more stated tasks or functions. As used herein, a module or component can exist on a hardware component independently of other modules or components. Alternatively, a module or component can be a shared element or process of other modules, programs, or machines.

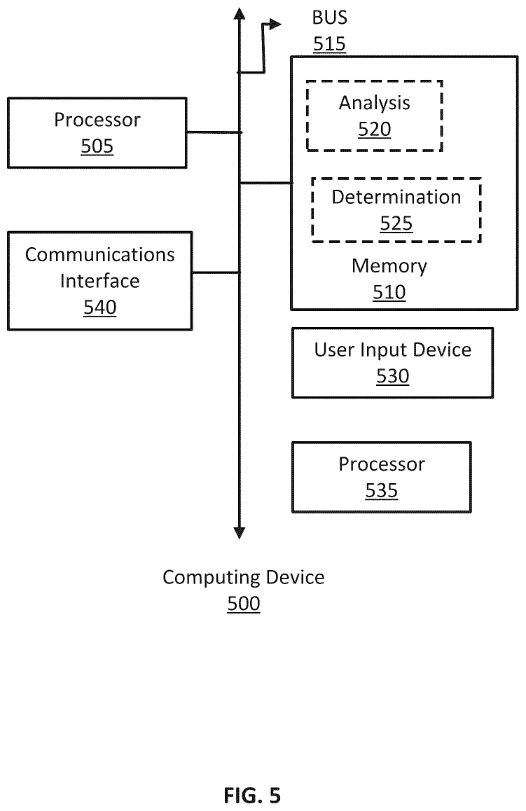

FIG. 5 shows a block diagram of an analysis system 500 used for sequencing a PKD1 gene. As illustrated in FIG. 5, modules, engines, or components (e.g., program, code, or instructions) executable by one or more processors may be used to implement the various subsystems of an analyzer system according to various embodiments. The modules, engines, or components may be stored on a non-transitory computer medium. As needed, one or more of the modules, engines, or components may be loaded into system memory (e.g., RAM) and executed by one or more processors of the analyzer system. In the example depicted in FIG. 5, modules, engines, or components are shown for implementing the methods or running any of the systems of the disclosure.

Thus, FIG. 5 illustrates an example computing device 500 suitable for use with systems and the methods according to this disclosure. The example computing device 500 includes a processor 505 which is in communication with the memory 510 and other components of the computing device 500 using one or more communications buses 515. The processor 505 is configured to execute processor-executable instructions stored in the memory 510 to perform one or more methods or operate one or more stations for sequencing a PKD1 gene according to different examples, such as those in FIG. 1-4 or 6, or disclosed elsewhere herein. In this example, the memory 510 may store processor-executable instructions 525 that can analyze 520 results for sample as discussed herein.

The computing device 500 in this example may also include one or more user input devices 530, such as a keyboard, mouse, touchscreen, microphone, etc., to accept user input. The computing device 500 may also include a display 535 to provide visual output to a user such as a user interface. The computing device 500 may also include a communications interface 540. In some examples, the communications interface 540 may enable communications using any of a variety of available protocols including without limitation TCP/IP (transmission control protocol/Internet protocol), SNA (systems network architecture), IPX (Internet packet exchange), AppleTalk®, and the like. Merely by way of example, network(s) 120 may be a local area network (LAN), networks based on Ethernet, Token-Ring, a wide-area network (WAN), the Internet, a virtual network, a virtual private network (VPN), an intranet, an extranet, a public switched telephone network (PSTN), an infra-red network, a wireless network (e.g., a network operating under any of the Institute of Electrical and Electronics (IEE®) 102.11 suite of protocols, Bluetooth®, and/or any other wireless protocol), and/or any combination of these and/or other networks. The computer-usable or computer-readable medium may be, but is not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random-access memory (RAM), including a magnetic RAM, core memory, magnetic disk storage mediums, optical storage mediums including optical fibers a read-only memory (ROM), an erasable programmable read-only memory (EPROM or Flash memory), a portable compact disc read-only memory (CD-ROM), and/or other computer-readable mediums for storing information. The computer-usable or computer-readable medium could even be paper or another suitable medium, upon which the program is printed, as the program can be electronically captured, via, for instance, optical scanning of the paper or other medium, then compiled, interpreted, or otherwise processed in a suitable manner if necessary, and then stored in a computer memory.

Computer program code for carrying out operations of the present disclosure may be written in an object-oriented programming language such as R®, JavaScript®, Java®, Java7®, Smalltalk®, Python®, LabVIEW®, C++®, MATLAB®, SQL®, or VisualBasic®. However, the computer program code for carrying out operations of the present disclosure may also be written in conventional procedural programming languages, such as the “C” programming language or even assembly language. The program code may execute entirely on the user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

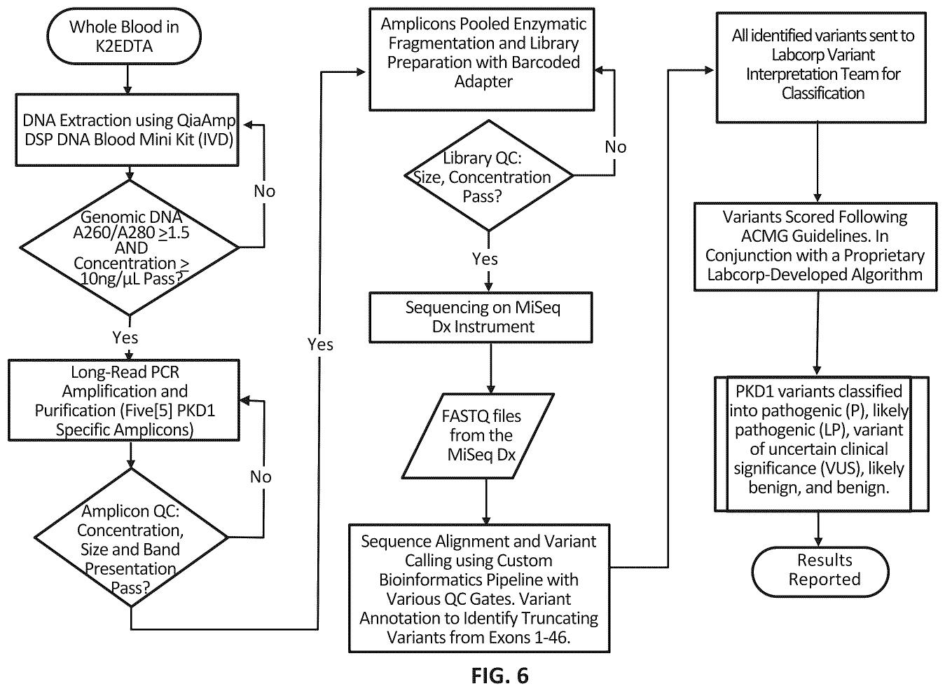

FIG. 6 shows an example workflow utilizing a system for long-range PCR amplification and sequencing of the PKD1 gene, including optional variant analysis steps that may be performed, in accordance with an embodiment of the disclosure. As shown in FIG. 6, a whole blood sample in di-potassium salt of ethylene-diamine-tetraacetic acid (K2EDTA) undergoes DNA extraction as described in the Examples below and elsewhere herein. Extraction is performed until a concentration of at least 10 ng/μL gDNA is reached and the ratio of absorbance at 260 nm and 280 nm (A260/A280) is greater than 1.5. The isolated gDNA then undergoes long-read PCR amplification as described herein for 5 fragments corresponding to exons 1-46 of the PKD1 gene. As noted herein, the first fragment may correspond to exon 1 of the PKD1 gene, the second fragment may correspond to exons 2-13 of the PKD1 gene, the third fragment may correspond to exons 14-21 of the PKD1 gene, the fourth fragment may correspond to exons 22-34 of the PKD1 gene, and the fifth fragment may correspond to exons 35-46 of the PKD1 gene. In certain embodiments, the primers used are those as described herein.

As shown in FIG. 6, after long-range PCR amplification, the amplicons are purified and may be subjected to a quality control evaluation for concentration, size, and band presentation. The amplicons are then pooled and undergo enzymatic fragmentation and library preparation with an adapter as described in the Examples and elsewhere herein. The library may be subjected to a quality control evaluation for size and concentration. Sequencing is then performed as described in the Examples and elsewhere wherein. Sequence alignment and variant calling may be performed to identify truncating variants from exons 1-46 of the PKD1 gene. PKD1 variants may be classified, for example, as pathogenic, likely pathogenic, variant of uncertain clinical significance, likely benign, or benign. Sequencing results are then reported, for example to a healthcare provider or the subject from which the whole blood sample was obtained.

Various embodiments of the disclosure have been described herein. It should be recognized that these embodiments are merely illustrative of the present disclosure. Variations of those preferred embodiments may become apparent to those of ordinary skill in the art upon reading the foregoing description. It is expected that skilled artisans can employ such variations as appropriate, and the disclosure is intended to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated or otherwise clearly contradicted by context.

### Examples

While the preferred embodiments of the invention have been illustrated and described, it will be appreciated that various changes can be made therein without departing from the spirit and scope of the invention. All printed patents and publications referred to in this application are hereby incorporated herein in their entirety by this reference.

### Example 1: Long-Range PCR Amplification of Exons 1-46 of the PKD1 Gene

All exons (1-46) of the PKD1 gene were sequenced using amplicon-based sequencing. The exons were enriched by long-range PCR. Five separate fragments of the PKD1 gene were generated per sample covering all 46 exons. Exons 1-46 were amplified as 5 fragments (or amplicons), where one fragment includes exon 35-46, a second fragment includes exons 22-34, a third fragment includes exons 14-21, a fourth fragment includes exons 2-13, and a fifth fragment corresponds to exon 1. Primer pairs were provided as 5 pools, one per fragment. Genomic DNA (gDNA) was extracted, normalized to 25 ng/μL, and 4 μL were added to 50 μL of the LC-PCR reaction.

Touchdown long-range PCR materials and methods include components for long-range PCR and touchdown long-range PCR cycling conditions. Components for long-range PCR include polymerase, forward (FW) and reverse (RV) primers, and PCR Cycling as shown in Table 1. The touchdown long-range PCR cycling conditions are summarized in Table 2.

TABLE 1
PCR cycling components and exemplary protocol

| Component | Protocol |
|---|---|
| Polymerase | LA Taq ® with dNTPs and GC Buffer I |
| | (TaKaRa) |
| Primers | 5 primer pairs as discussed in Table 3. |
| PCR Cycling | Touchdown long-range PCR |

TABLE 2
Touchdown (TD) long-range (LR)-PCR Reaction Conditions
TD long-range PCR La Taq

| | | |
|---|---|---|
| Temperature (° C.) | Time | # of Cycles |

| | | | |
|---|---|---|---|
| 98 | 3 | min | 1 |
| 98 | 10 | sec | 20 |
| 70-50 (1 degree reduction | 15 | sec | |
| every cycle) | | | |
| 69 | 10 | min | |
| 98 | 10 | sec | 15 |
| 50 | 15 | sec | |
| 69 | 10 | min | |
| 69 | 10 | min | 1 |

| | | |
|---|---|---|
| 4 | Hold | 1 |

A list of primers used for amplifying exons 1-46 are shown in Table 3 as SEQ ID NOs: 15-16, 9-10, and 19-24. SEQ ID NOs: 15-16 were designed for amplifying Exon 1 of PKD1. SEQ ID NOs: 9-10 were designed for amplifying Exons 2-13 of PKD1. SEQ ID NOs: 19-20 were designed for amplifying Exons 14-21 of PKD1. SEQ ID NOs: 21-22 were designed for amplifying Exons 22-34 of PKD1. SEQ ID NOs: 23-24 were designed for amplifying Exons 35-46 of PKD1.

TABLE 3
Primer pairs and sequences

| | SEQ | | |
|---|---|---|---|
| | ID | | long-range PCR Primer |
| Exons | NO: | Strand* | sequence (5′-3′)** |
| 1 | 15 | FW | GAGGCTGCGGGTACTGACTC |
| | 16 | RV | GGCTTCTCAAAGAGCCTCAATTTC |
| 2-13 | 9 | FW | TGTCGTGTAAGTTGCTAGTGCT |
| | 10 | RV | TGAACCGGGACAGGGGTG |
| 14-21 | 19 | FW | TTTCCCTGTCTGTTGGGAGGTAACT |
| | 20 | RV | CCTGCGTTCACACAGGACAGAAC |
| 22-34 | 21 | FW | ATGTGAAGAGGTGCCTTGTGTGGT |
| | 22 | RV | TTAAAAACCCGCCCATAATTTCTC |
| | | | ACTGC |
| 35-46 | 23 | FW | GCAGGCTCATGGGGCTTTGTAGGAGCAG |
| | 24 | RV | GCACAGCCCGCTGTACCTGAGGACT |

The amplification reaction mixture was put together as outlined in Table 4 to create the master amplification mix. To make the master mixes, genomic DNA (gDNA) samples were normalized to 25 ng/μl and the five primer pairs of Table 3 added to a final concentration of 4 μM.

TABLE 4
Master amplification mixes for each primer pair.

| | Volume (μL) | |
|---|---|---|
| Reagents | per reaction | 15% Overage (μL) |
| 2X GC Buffer I | 25 | 28.75 |
| dNTP Mixture (2.5 mM each) | 8 | 9.2 |
| TaKaRa LA Taq ® | 0.5 | 0.575 |
| Water | 10.5 | 12.075 |

On the appropriate well of the reaction plate 48.4 μL of master mix was aliquoted. To each aliquoted master mixes, 2.2 μL of primer sets were added (including 10% overage). 46 μL of master mix was added to each sample well with 4 μL of template gDNA was added. 4 μL of positive control was added to one experimental well, and 4 μL water was added to the no template control well. The conditions for the PCR reaction were setup as summarized in Table 5.

TABLE 5
Touchdown (TD) long range (LR)-PCR Reaction Conditions
TD long-range PCR LA Taq

| | | |
|---|---|---|
| Temperature (° C.) | Time | # of Cycles |

| | | | |
|---|---|---|---|
| 98 | 3 | min | 1 |
| 98 | 10 | sec | 20 |
| 70-50 (1 degree reduction | 15 | sec | |
| every cycle) | | | |
| 69 | 10 | min | |
| 98 | 10 | sec | 15 |
| 50 | 15 | sec | |
| 69 | 10 | min | |
| 69 | 10 | min | 1 |

| | | |
|---|---|---|
| 4 | Hold | 1 |

The workflow of the amplification method for exons 1-46 is outlined in FIGS. 1-2 and 6.

Following long-range PCR, the five amplicons were pooled together with equal molarity and impurities were removed in a first purification step using magnetic beads. Specifically, long-range PCR products were purified using PKD1 DNA Purification Beads and washed with 80% ethanol. The purified bead-long-range PCR product pellet was then visualized using gel electrophoreses on a TapeStation® System (Agilent, Santa Clara, CA) for quality control. The long-range PCR products were quantified by fluorescence (Qubit dsDNA BR Assay Kit and Quant-iT dsDNA Broad-Range Assay Kit).

Next, the purified pool of amplicons was sheared with enzyme mix (Twist Bioscience®, South San Francisco, CA) and any nicks in anti-double stranded DNA (dsDNA) were repaired according to the following enzymatic fragmentation master mix: 25 μL water (27.5 μL with 10% overage) and 5 μL PKD1 Fragmentation Buffer (5.5 μL with 10% overage) were vortexed and briefly spun down, then 10 μL (11 μL with 10% overage) PKD1 Fragmentation Enzyme was added and mixed by pipetting. 40 μL enzymatic fragmentation master mix was plated on a 96 well plate and 10 μL of each sample pool was added and mixed by pipetting. The mixture was thermal cycled according to the following: 32° C. for 7 minutes, 65° C. for 30 minutes, and held at 4° C. when complete.

The product was then poly(dA) tailed. 5 μL PKD1 Adapters were added to each sample well containing poly(dA)-tailed DNA, and mixed by pipetting. The ligation master mix was prepared as follows: 15 μL water (16.5 μL with 10% overage) and 20 μL PKD1 Ligation Buffer (22 μL with 10% overage) were vortexed and briefly spun down, then 10 μL (11 μL with 10% overage) PKD1 Ligation Mix was added and mixed by pipetting. 45 μL of the ligation master mix was added to the samples and mixed by pipetting. The ligation reaction was incubated at 20° C. for 15 minutes in the thermal cycler, then spun down and stored at room temperature. The samples were then purified using PKD1 DNA purification beads according to the methods described above.

Finally, after the adapter was attached to the fragment, the product was amplified with a universal primer and placed in the thermal cycler to generate a library as shown in Table 6, below. 10 μL of primers from the PKD1 UDI Primer Plate was added to each of the libraries, and mixed by pipetting. 25 μL of PKD1 Amplification Mix (described above in Table 4) was added and mixed by pipetting.

TABLE 6
Library generation conditions

| Step | Temperature | Time | | Number of Cycles |
|---|---|---|---|---|
| Initialization | 98° C. | 45 | seconds | 1 |
| Duration | 98° C. | 15 | seconds | 6-8* |
| Annealing | 60° C. | 30 | seconds | |
| Extension | 72° C. | 30 | seconds | |
| Final Extension | 72° C. | 1 | minute | 1 |

| | | | |
|---|---|---|---|
| Final Hold | 4° C. | HOLD | 4 |

*6-8 cycles recommended when starting with 50-100 ng of high quality gDNA

Once the library was generated, a second purification step using the PKD1 DNA Purification Beads was performed as described above. Quantification was conducted by fluorescence using a Qubit® or Quant-IT® Assay Kit (Invitrogen®, Waltham, MA). Using the TapeStation® System (Agilent®, Santa Clara, CA), quality control and quantification was also performed. Sequencing was performed using an Illumina MiSeq® System (Illumina®, San Diego, CA). The workflow of the long-range amplification and sequencing assay for sequencing the PKD1 gene is outlined in FIGS. 1-2 and 6.

### Example 2: Dual Amplification and Sequencing Assay Long-Range PCR Amplification of PKD1 Exons 1-33 and Target-Enrichment PCR for PKD1 Exons 34-46

Initial primers tested for long-range PCR of exons 1-33 are shown in Table 7 as SEQ ID NOs: 1-18. SEQ ID NOs: 1-4 primers were designed for amplifying Exons 22-33 of PKD1. SEQ ID NOs: 5-8 primers were designed for amplifying Exons 14-21 of PKD1. SEQ ID NOs: 9-12 primers were designed for amplifying Exons 2-13 of PKD1. SEQ ID NOs: 13-18 primers were designed for amplifying Exon 1.

TABLE 7
Primer pairs and sequences

| | SEQ | | |
|---|---|---|---|
| | ID | | long-range PCR Primer |
| Exons | NO: | Strand* | sequence (5′-3′)** |
| 22-33 | 1 | FW | TGAGGACCCGTGTAGAGAGG |
| | 2 | RV | TGTGCGTGACTACATACAAGG |
| 22-33 | 3 | FW | GCTTAGTGAGGAGGCTGTGG |
| | 4 | RV | TGTGCGTGACTACATACAAGGTA |
| 14-21 | 5 | FW | TTTCTGAGCCTCGGTTTCCC |
| | 6 | RV | AACGGCTGAGGCTACTGAAG |
| 14-21 | 7 | FW | TCGGTTTCCCTGTCTGTTGG |
| | 8 | RV | GAACGGCTGAGGCTACTGAA |
| 2-13 | 9 | FW | TGTCGTGTAAGTTGCTAGTGCT |
| | 10 | RV | TGAACCGGGACAGGGGTG |
| 2-13 | 11 | FW | TGTCGTGTAAGTTGCTAGTGC |
| | 12 | RV | TGAACCGGGACAGGGGT |
| 1 | 13 | FW | GCTGCGGGTACTGACTCG |
| | 14 | RV | GCCGTGATGAGGTATGGAG |
| 1 | 15 | FW | GAGGCTGCGGGTACTGACTC |
| | 16 | RV | GGCTTCTCAAAGAGCCTCAATTTC |
| 1 | 17 | FW | ATCCACACCGCGGAAGAAGG |
| | 18 | RV | CCGTGATGAGGTATGGAGAGACAA |

For long-range PCR of exons 1-33, the amplification reaction mixture was prepared as outlined in Table 8 as a master mix. To make the master mixes, genomic DNA (gDNA) samples were normalized to 50 ng/μl and the separate primer pairs of Table 7 added for a final concentration of 10 μM for each primer.

Table 8. Master mixes for each primer pair.

TABLE 8
Master mixes for each primer pair.

| | Volume (μL) | |
|---|---|---|
| Reagents | per reaction | 10% Overage (μL) |
| 2X GC Buffer I | 25 | 27.5 |
| dNTP Mixture (2.5 mM each) | 8 | 8.8 |
| Forward Primer (10 μM) | 1 | 1.1 |
| Reverse Primer (10 μM) | 1 | 1.1 |
| TaKaRa LA Taq ® | 0.5 | 0.55 |
| Water | 12.5 | 13.75 |

On the appropriate well of the reaction plate 48 μL of master mix was added. For each primer pair, 2 μL of template (i.e., 100 ng) was added. The conditions for the PCR are summarized in Table 9.

TABLE 9
Touchdown (TD) long-range PCR Reaction Conditions
TD long-range PCR LA Taq

| | | |
|---|---|---|
| Temperature (° C.) | Time | # of Cycles |

| | | | |
|---|---|---|---|
| 98 | 3 | min | 1 |
| 98 | 10 | sec | 20 |
| 70-50 (1 degree reduction | 15 | sec | |
| every cycle) | | | |
| 69 | 10 | min | |
| 98 | 10 | sec | 15 |
| 50 | 15 | sec | |
| 69 | 10 | min | |
| 69 | 10 | min | 1 |

| | | |
|---|---|---|
| 4 | Hold | 1 |

The workflow of the amplification method for exons 1-33 is outlined in FIG. 3.

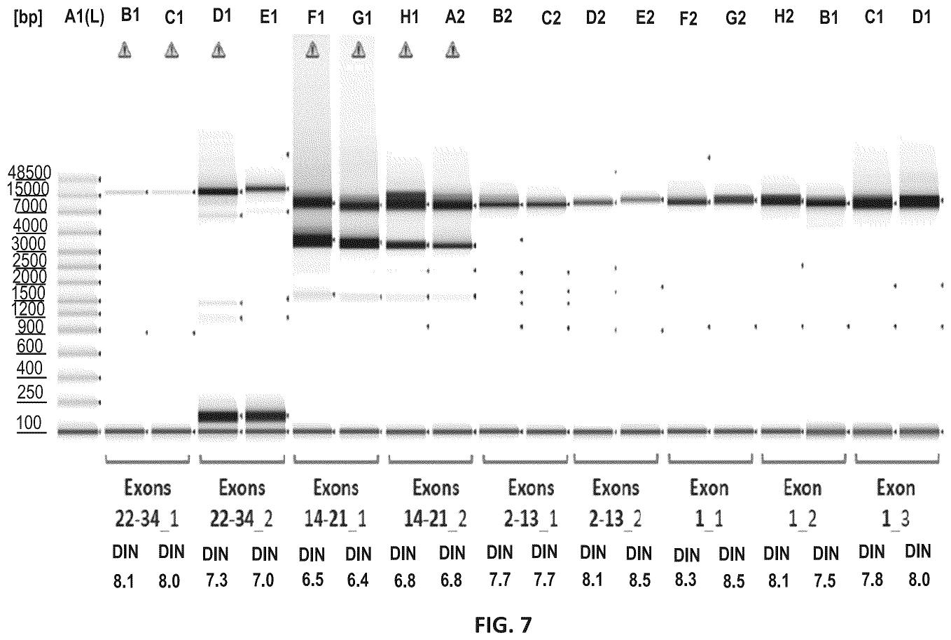

Following long-range PCR, the PCR products were run on a TapeStation® System (Agilent®, Santa Clara, CA) for quality control and quantification. As exemplified in FIG. 7, showing two gDNA samples obtained from blood specimens, all designs (i.e., primer pairs) for Exons 2-13 worked well with single, strong bands present. The first design for Exons 22-33 (i.e., SEQ ID NOs: 1 and 2) had a single faint band and the second design 34 (i.e., SEQ ID NOs: 3 and 4) for Exons 22-33 had a strong target band but also had a strong small band, possibly due to the formation primer dimers. Both designs of Exons 2-13 resulted in two strong bands, possibly indicating off-target amplification. Based on these results, SEQ ID NOs: 15 and 16 were chosen for long-range PCR amplification of exon 1, SEQ ID NOs: 9 and 10 were chosen for long-range PCR amplification of exons 2-13, while new primers were developed for long-range PCR amplification of exons 14-21 and 22-33 (see Example 1).

Once the PCR products were amplified, the four amplicons were pooled together with equal molarity and impurities were removed in a first purification step using magnetic beads. Next, the purified pool of amplicons was sheared with enzyme mix (Twist Bioscience®, South San Francisco, CA) and any nicks in anti-double stranded DNA (dsDNA) were repaired. The product was then poly(dA) tailed. Finally, an adapter was attached to the fragment and the product was amplified with a universal primer for a few cycles to generate a library. Adapter ligation and library generation was performed as described above in Example 1. An example of PCR amplification is shown in Table 10, below.

TABLE 10
Library generation conditions

| Step | Temperature | Time | | Number of Cycles |
|---|---|---|---|---|
| Initialization | 98° C. | 45 | seconds | 1 |
| Duration | 98° C. | 15 | seconds | 6-8* |
| Annealing | 60° C. | 30 | seconds | |
| Extension | 72° C. | 30 | seconds | |
| Final Extension | 72° C. | 1 | minute | 1 |

| | | | |
|---|---|---|---|
| Final Hold | 4° C. | HOLD | — |

*6-8 cycles recommended when starting with 50-100 ng of high quality gDNA

Once the library was generated, a second purification step using the TapeStation® System (Agilent®, Santa Clara, CA) for quality control and quantification was carried out. Quantification was conducted by fluorescence using a Qubit® or Quant-IT® Assay Kit (Invitrogen®, Waltham, MA) before sequencing. Sequencing was carried out using an Illumina MiSeq® System (Illumina®, San Diego, CA). The workflow of the dual amplification and sequencing assay for sequencing the PKD1 gene is outlined in FIG. 3.

Target-enrichment was conducted in collaboration with Twist Biosciences® (South San Francisco, CA). Target-enrichment was done by designation of 120 bp probes, as shown in Table 11. For target-enrichment PCR, gDNA was sheared with enzyme mix (Twist Bioscience®, South San Francisco, CA) and any nicks in anti-double stranded DNA (dsDNA) were repaired. A tailing of the product followed. Next, an adapter was attached to the fragment and the product was amplified with a universal primer for a few cycles to generate a library. Following, the library was hybridized with a probe panel overnight. The next day, the hybridized pool was washed to remove any off-target sequence. The enriched library was purified using magnetic beads and prepare for quality control and sequencing. Exons 34-46 of the PKD1 gene were sequenced using the probe enrichment product. Sequencing was carried out using an Illumina MiSeq® System (Illumina®, San Diego, CA). A list of probes identified by their chromosome and chromosome coordinates can be found in Table 11.

TABLE 11
For probe enrichment of Exons 34-46

| Chromosome | Chromosome Coordinates | | Chromosome | Chromosome Coordinates | |
|---|---|---|---|---|---|
| chr16 | 2089705 | 2089825 | chr16 | 2092993 | 2093113 |
| chr16 | 2089817 | 2089937 | chr16 | 2093334 | 2093454 |
| chr16 | 2089928 | 2090048 | chr16 | 2093441 | 2093561 |
| chr16 | 2090040 | 2090160 | chr16 | 2093549 | 2093669 |
| chr16 | 2090161 | 2090281 | chr16 | 2093656 | 2093776 |
| chr16 | 2090273 | 2090393 | chr16 | 2093777 | 2093897 |
| chr16 | 2090385 | 2090505 | chr16 | 2093888 | 2094008 |
| chr16 | 2090497 | 2090617 | chr16 | 2093999 | 2094119 |
| chr16 | 2090618 | 2090738 | chr16 | 2094110 | 2094230 |
| chr16 | 2090730 | 2090850 | chr16 | 2097127 | 2097247 |
| chr16 | 2090841 | 2090961 | chr16 | 2097219 | 2097339 |
| chr16 | 2090953 | 2091073 | chr16 | 2097311 | 2097431 |
| chr16 | 2091074 | 2091194 | chr16 | 2097403 | 2097523 |
| chr16 | 2091402 | 2091522 | chr16 | 2088687 | 2088807 |
| chr16 | 2091497 | 2091617 | chr16 | 2088803 | 2088923 |
| chr16 | 2091760 | 2091880 | chr16 | 2088919 | 2089039 |
| chr16 | 2091869 | 2091989 | chr16 | 2089035 | 2089155 |
| chr16 | 2091979 | 2092099 | chr16 | 2089156 | 2089276 |
| chr16 | 2092088 | 2092208 | chr16 | 2089272 | 2089392 |
| chr16 | 2092459 | 2092579 | chr16 | 2089388 | 2089508 |
| chr16 | 2092561 | 2092681 | chr16 | 2089504 | 2089624 |
| chr16 | 2092663 | 2092783 | chr16 | 2089625 | 2089745 |
| chr16 | 2092765 | 2092885 | chr16 | 2092310 | 2092430 |
| chr16 | 2092886 | 2093006 | chr16 | 2094913 | 2095033 |

### Example 3: Embodiments

A1. A method of sequencing a PKD1 gene, the method comprising
   (a) amplifying exons 1-46 of the PKD1 gene using long-range PCR to form a plurality of long-range PCR products; and
   (b) sequencing the plurality of long-range PCR products.
A2. The method of embodiment A1, further comprising purifying the plurality of long-range PCR products prior to the sequencing step.
A3. The method of embodiment A1 or A2, further comprising pooling the plurality of long-range PCR products prior to the sequencing step.
A4. The method of any one of embodiments A1-A3, further comprising shearing the plurality of long-range PCR products prior to the sequencing step.
A5. The method of any one of embodiments A1-A4, further comprising amplifying the plurality of long-range PCR products prior to the sequencing step.
A6. The method of any one of embodiments A1-A5, further comprising creating a library of the long-range PCR products.
A7. The method of any one of embodiments A1-A6, wherein the long-range PCR comprises contacting the PKD1 gene with a plurality of primers comprising the nucleic acid sequences of SEQ ID NOs: 9-10, 15-16, and 19-24.
A8. The method of any one of embodiments A1-A7, the long-range PCR comprises amplifying exons 1-46 of the PKD1 gene as five fragments.
A9. The method of embodiment A8, wherein the first fragment corresponds to exon 1 of the PKD1 gene, the second fragment corresponds to exons 2-13 of the PKD1 gene, the third fragment corresponds to exons 14-21 of the PKD1 gene, the fourth fragment corresponds to exons 22-34 of the PKD1 gene, and the fifth fragment corresponds to exons 35-46 of the PKD1 gene.
A10. A composition comprising a plurality of primers for conducting the method of any one of embodiments A1-A9.
A11. The composition of embodiment A10, wherein the plurality of primers primarily amplify non-pseudogene forms of the PKD1 gene.
A12. The composition of embodiment A10 or A11, wherein the plurality of primers comprise the nucleic acid sequences of SEQ ID NOs: 9-10, 15-16, and 19-24.
B1. A kit comprising a plurality of primers comprising the nucleic acid sequences of SEQ ID NOs: 9-10, 15-16, and 19-24 and instructions for use.
C1. A system for conducting the method of any one of embodiments A1-A9, the system comprising:
   a. a component for amplifying exons 1-46 of the PKD1 gene by long-range PCR to form a plurality of long-range PCR products; and
   b. a component for sequencing the plurality of long-range PCR products.
D1. A method of sequencing a PKD1 gene, the method comprising:
   a. amplifying exons 1-33 of the PKD1 gene using long-range PCR to form a plurality of long-range PCR products,
   b. amplifying exons 34-46 of the PKD1 gene using target-enrichment PCR to form a plurality of target-enrichment PCR products; and
   c. sequencing the plurality of long-range PCR products and the plurality of target-enrichment PCR products.
D2. The method of embodiment D1, further comprising purifying the plurality of long-range PCR products and/or the plurality of target-enrichment PCR products prior to the sequencing step.
D3. The method of any one of embodiments D1 or D2, further comprising pooling the plurality of long-range PCR products prior to the sequencing step.
D4. The method of any one of embodiments D1-D3, further comprising shearing the plurality of long-range PCR products and/or the plurality of target-enrichment PCR products prior to the sequencing step.
D5. The method of any one of embodiments D1-D4, further comprising amplifying the plurality of long-range PCR products and/or the plurality of target-enrichment PCR products prior to the sequencing step.
D6. The method of any one of embodiments D1-D5, wherein the long-range PCR comprises contacting the PKD1 gene with a plurality of primers comprising the nucleic acid sequences of SEQ ID NOs: 1-18.
D7. The method of any one of embodiments D1-D6, wherein the long-range PCR of exons 1-33 comprises amplifying exons 1-33 of the PKD1 gene as four fragments.
D8. The method of embodiment D7, wherein the first fragment corresponds to exon 1 of the PKD1 gene, the second fragment corresponds to exons 2-13 of the PKD1 gene, the third fragment corresponds to exons 14-21 of the PKD1 gene, and the fourth fragment corresponds to exons 22-33 of the PKD1 gene.
D9. The method of any of embodiments D1-D8, wherein the target-enrichment PCR comprises
   a. contacting the PKD1 gene with a plurality of probes, wherein exons 34-46 hybridize to the plurality of probes; and
   b. purifying the hybridized exons 34-46.
D10. The method of embodiment D9, wherein the plurality of probes comprise the nucleic acid sequences corresponding the chromosome coordinates of Table 11.
E1. A composition comprising a plurality of primers for conducting the method of any one of embodiments D1-D10.
E2. The composition of embodiment E1, wherein the plurality of primers and probes primarily amplify non-pseudogene forms of the PKD1 gene.
E3. The composition of embodiment E1 or E2, wherein the plurality of primers comprise the nucleic acid sequences of SEQ ID NOs: 1-18.
E4. The composition of any one of embodiments E1-E3, wherein the plurality of probes comprise the nucleic acid sequences corresponding the chromosome coordinates of Table 11.
F1. A kit comprising primers comprising any one of SEQ ID NOs: 1-18 and/or any one of the plurality of probes comprising nucleic acid sequences corresponding the chromosome coordinates of Table 11 and instructions for use.
F2. A system for conducting the method of any one of embodiments D1-D10, the system comprising:
   a. a component for amplifying exons 1-33 of the PKD1 gene by long-range PCR to form a plurality of long-range PCR products,
   b. a component for amplifying exons 34-46 of the PKD1 gene using target-enrichment PCR to form a plurality of target-enrichment PCR products; and
   c. a component for sequencing the plurality of long-range PCR products and the plurality of target-enrichment PCR products.

The disclosed can be better understood by reference to the following claims.

What is claimed is:

1. A method of sequencing a PKD1 gene, the method comprising
amplifying exons 1-46 of the PKD1 gene using long-range PCR to form a plurality of long-range PCR products; and
sequencing the plurality of long-range PCR products.

2. The method of claim 1, further comprising purifying the plurality of long-range PCR products prior to the sequencing step.

3. The method of claim 1, further comprising pooling the plurality of long-range PCR products prior to the sequencing step.

4. The method of claim 1, further comprising shearing the plurality of long-range PCR products prior to the sequencing step.

5. The method of claim 1, further comprising amplifying the plurality of long-range PCR products prior to the sequencing step.

6. The method of claim 1, further comprising creating a library of the long-range PCR products.

7. The method of claim 1, wherein the long-range PCR comprises contacting the PKD1 gene with a plurality of primers comprising the nucleic acid sequences of SEQ ID NOs: 9-10, 15-16, and 19-24.

8. The method of claim 1, the long-range PCR comprises amplifying exons 1-46 of the PKD1 gene as five fragments.

9. The method of claim 8, wherein the first fragment corresponds to exon 1 of the PKD1 gene, the second fragment corresponds to exons 2-13 of the PKD1 gene, the third fragment corresponds to exons 14-21 of the PKD1 gene, the fourth fragment corresponds to exons 22-34 of the PKD1 gene, and the fifth fragment corresponds to exons 35-46 of the PKD1 gene.

10. A composition comprising a plurality of primers for conducting the method of claim 1.

11. The composition of claim 10, wherein the plurality of primers primarily amplify non-pseudogene forms of the PKD1 gene.

12. The composition of claim 10, wherein the plurality of primers comprise the nucleic acid sequences of SEQ ID NOs: 9-10, 15-16, and 19-24.

13. The composition of claim 12, wherein the plurality of primers comprise the nucleic acid sequences of SEQ ID NOS: 15 and 16 for amplification of exon 1 of the PKD1 gene, SEQ ID NOs: 9 and 10 for amplification of exons 2-13 of the PKD1 gene, SEQ ID NOs: 19 and 20 for amplification of exons 14-21 of the PKD1 gene, SEQ ID NOs: 21 and 22 for amplification of exons 22-34 of the PKD1 gene, and SEQ ID NOs: 23 and 24 for amplification of exons 35-46 of the PKD1 gene.

14. A kit comprising a plurality of primers comprising the nucleic acid sequences of SEQ ID NOs: 9-10, 15-16, and 19-24 and instructions for use.

15. A system for conducting the method of claim 1, the system comprising:
a component for amplifying exons 1-46 of the PKD1 gene by long-range PCR to form a plurality of long-range PCR products; and
a component for sequencing the plurality of long-range PCR products.
